(19) United States
(12) Patent Application Publication
(10) Pub. No.: US 2020/0323454 A1
(43) Pub. Date: Oct. 15, 2020

(54) CARDIAC TRAJECTORY CURVE ANALYSIS FOR CLINICAL DECISION MAKING AND ANALYSIS

(71) Applicant: Siemens Healthcare GmbH, Erlangen (DE)

(72) Inventors: Viorel Mihalef, North Brunswick, NJ (US); Puneet Sharma, Princeton Junction, NJ (US)

(21) Appl. No.: 16/381,131

(22) Filed: Apr. 11, 2019

(51) Int. Cl. A61B 5/04; G16H 50/70; A61B 5/00

(52) U.S. Cl. CPC A61B 5/04012; G16H 50/70; G16H 15/00; A61B 5/0035; A61B 5/7203

(57) ABSTRACT

Systems and methods are provided for cardiac trajectory curve analysis for supporting clinical decision making and analysis. One or more trajectory curves representing cardiac movement are generated. Regions of the one or more trajectory curves that correspond to cardiac events are identified. Features of interest associated with the identified regions are determined. A correspondence map is generated by mapping the determined features of interest to clinical parameters.

### TECHNICAL FIELD

The present invention relates generally to cardiac trajectory curve analysis for clinical decision making and analysis, and more particularly to extracting local features of interest from trajectory curves for improved clinical decision making and analysis.

### BACKGROUND

In the current clinical practice, global cardiac measures are traditionally extracted from medical images to evaluate the quality of the medical images and provide functional analysis of the medical images. Global cardiac measures refer to measures of the heart, or chambers of the heart, as a whole. Examples of global cardiac measure include stroke volume (SV), ejection fraction (EF), and global longitudinal strain (GLS). Such global cardiac measures may be inadequate when compared to local or regional cardiac measures to detect data quality limitations or provide functional estimates that may help with patient stratification or treatment planning. Further, such global cardiac measures do not take into account regional characteristics, such as differential contraction due to the anatomic configuration of the ventricular myocardial band. Despite the drawbacks of using such global cardiac measures, the use of local cardiac measures has traditionally been limited. This is in part because the variability of traditional local cardiac measures makes it harder to be included in a clinical decision standard.

### BRIEF SUMMARY OF THE INVENTION

In accordance with one or more embodiments, local features of interest are extracted from cardiac trajectory curves for supporting clinical decision making and analysis. One or more trajectory curves representing cardiac movement are generated. Regions of the one or more trajectory curves that correspond to cardiac events are identified. Features of interest associated with the identified regions are determined. A correspondence map is generated by mapping the determined features of interest to clinical parameters.

In one embodiment, the trajectory curves may include an endocardial trajectory curve, a myocardial trajectory curve, and an epicardial trajectory curve. The regions on the trajectory curves may include geometric regions that correspond to cardiac events, such as, e.g., a beginning of systole, a beginning a diastole, a middle of diastole, and an A-wave. Features of interest associated with the regions may include geometric measures and anatomical measures.

In one embodiment, the correspondence map may be generated by mapping the trajectory curves and/or the determined features of interest to the clinical parameters based on a statistical mapping of the trajectory curves and/or the determined features of interest to the clinical parameters. For example, the statistical mapping may be a correlation between the trajectory curves and/or the determined features of interest and the clinical parameters. Mapping the trajectory curves and/or the determined features of interest to the clinical parameters may be performed using a machine learning model. The correspondence map may be used for clinical decision making. In one embodiment, the clinical parameters may be visually represented on a heart unravelling image.

These and other advantages of the invention will be apparent to those of ordinary skill in the art by reference to the following detailed description and the accompanying drawings.

### BRIEF DESCRIPTION OF THE DRAWINGS

FIG. 1 shows a cross section cine magnetic resonance image with endocardial trajectory curves;

FIG. 2 shows a high-level workflow for cardiac trajectory curve analysis for clinical decision making and analysis, in accordance with one or more embodiments;

FIG. 3 shows a method for cardiac trajectory curve analysis for clinical decision making and analysis stage, in accordance with one or more embodiments;

FIG. 4 shows an exemplary endocardial trajectory curve;

FIG. 5 shows an exemplary ventricular myocardial band;

FIG. 6 shows an exemplary heart unravelling image having trajectory curves mapped thereon, in accordance with one or more embodiments; and

FIG. 7 shows a high-level block diagram of a computer.

### DETAILED DESCRIPTION

The present invention generally relates to methods and systems for cardiac trajectory curve analysis for clinical decision making and analysis. Embodiments of the present invention are described herein to give a visual understanding of such systems and methods. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Further, it should be understood that while the embodiments discussed herein may be discussed with respect to cardiac trajectory curve analysis for clinical decision making and analysis, the present invention is not so limited. Embodiments of the present invention may be applied for any type of analysis of trajectory curves for any type of decision making and analysis.

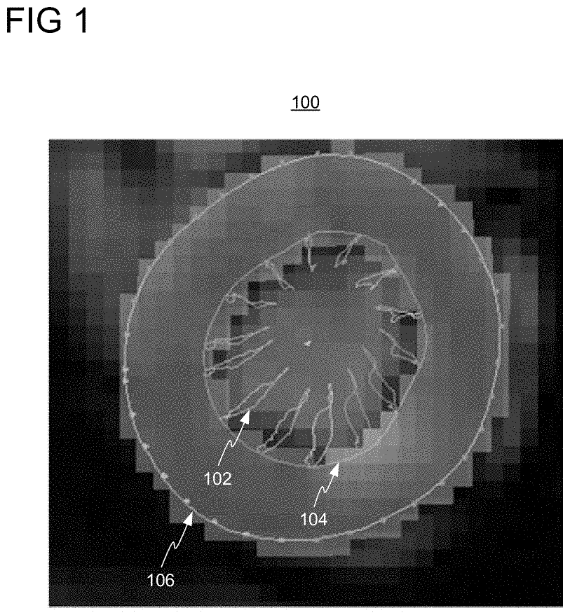

FIG. 1 shows a cross sectional cine magnetic resonance (MR) image 100 of a base of a left ventricle of a heart for a cardiac cycle. Image 100 shows a segmented endocardium 104 and epicardium 106 and includes a plurality of endocardial trajectory curves 102. Endocardial trajectory curves 102 represent the movement (e.g., contraction and relaxation) of the endocardium over a cardiac cycle. Similar trajectory curves may be computed for, e.g., the myocardium, the epicardium, and other objects of interest. Such trajectory curves may be computed according to, e.g., an optical flow tracking algorithm, a speckle flow tracking algorithm, or any other known approach.

Embodiments of the present invention extract features of interest from trajectory curves, and generates correspondence maps associating the extracted features of interest to clinical parameters. Such features of interest extracted from regions of the trajectory curves account for local regional cardiac characteristics, such as, e.g., differential contraction due to the anatomic configuration of the ventricular myocardial band, that global cardiac measures do not account for. Advantageously, such correspondence maps support improved clinical decision making and analysis for, e.g., medical image quality evaluation, patient stratification, and diagnostic, planning, or intervention support with greater accuracy.

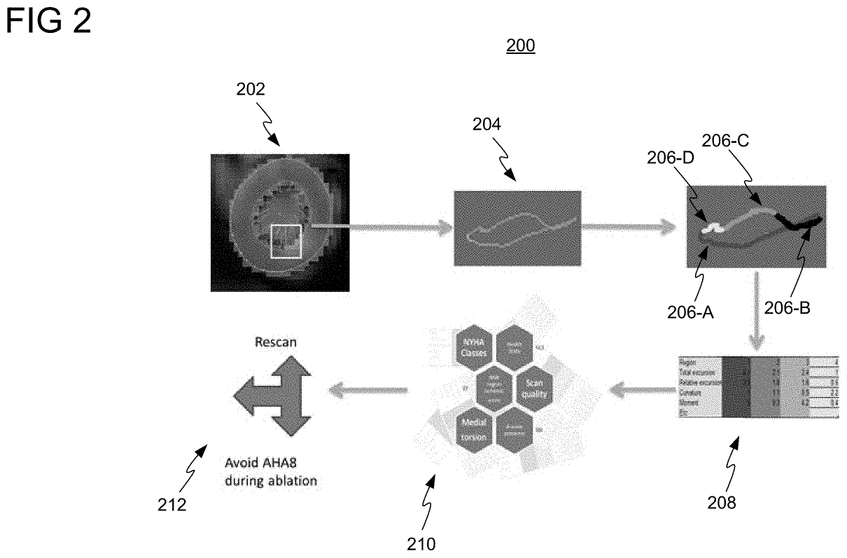

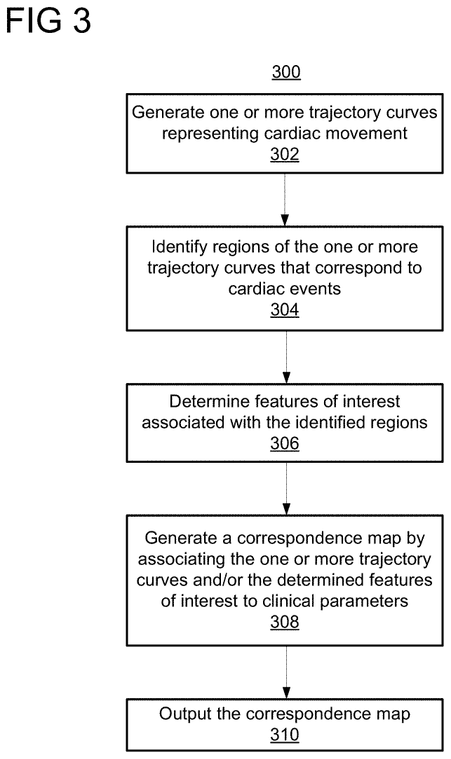

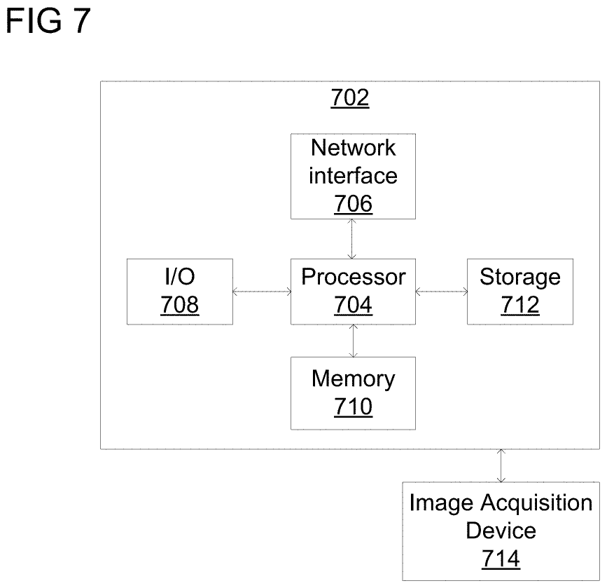

FIG. 2 shows a high-level workflow 200 for cardiac trajectory curve analysis for clinical decision making and analysis, in accordance with one or more embodiments. Workflow 200 of FIG. 2 will be described with reference to FIG. 3. FIG. 3 shows a method 300 for cardiac trajectory curve analysis for clinical decision making and analysis, in accordance with one or more embodiments. The steps of method 300 may be performed by any suitable computing device, such as, e.g., computer 702 of FIG. 7.

At step 302, one or more trajectory curves representing cardiac movement are generated. The one or more trajectory curves may be trajectory curve 204 shown in FIG. 2. Trajectory curve 204 may be, for example, an endocardial trajectory curve representing movement of the endocardium, a myocardial trajectory curve representing movement of the myocardium, an epicardial trajectory curve representing movement of the epicardium, or any other type of trajectory curve.

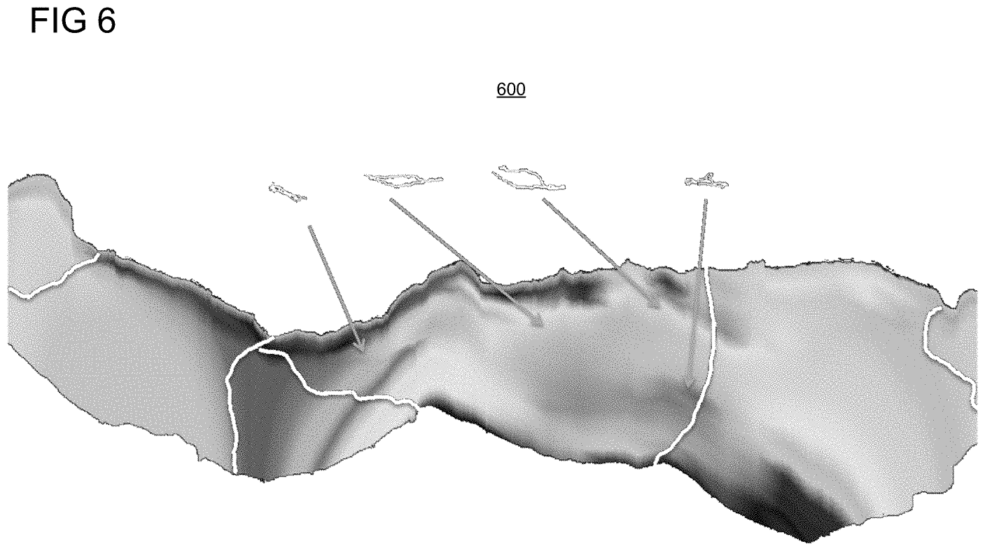

In one embodiment, trajectory curve 204 may be generated based on, e.g., an optical flow tracking algorithm, a speckle flow tracking algorithm, or any other known approach. Image 202 is based on a cine MR medical image in FIG. 2, but may be based on a medical image of any suitable modality, such as, e.g., computed tomography (CT), ultrasound (US), or any other suitable modality or combination of modalities. The medical image from which image 202 is based on may be received directly from an image acquisition device (e.g., image acquisition device 614 of FIG. 6) or may be received by loading previously stored images acquired using the image acquisition device. In another embodiment, trajectory curve 204 is a previously generated trajectory curve received from a computing device.

At step 304, regions of the one or more trajectory curves that correspond to cardiac events are identified. The regions may be region 1 206-A, region 2 206-B, region 3 206-C, and region 4 206-D (collectively referred to as regions 206) of trajectory curve 204, as shown in FIG. 2. Regions 206 may correspond to any cardiac event. In one embodiment, region 1 206-A corresponds to the beginning of ventricular systole, region 2 206-B corresponds to the beginning of ventricular diastole, region 3 206-C corresponds to middle of ventricular diastole, and region 4 206-D corresponds to the A-wave. Other cardiac events are also contemplated. For example, regions of the trajectory curve may be classified according to “global” stages (e.g., atrial (or ventricular) systole or diastole), sub-stages (e.g., iso-volumic contraction or iso-volumic relaxation), rapid contract (as the first phase of ventricular systole), or to rapid filling (as the first stage of ventricular diastole). In other examples, pathological events (e.g., impaired contraction during ventricular or atrial fibrillation) can be tagged to regions of the trajectory curve.

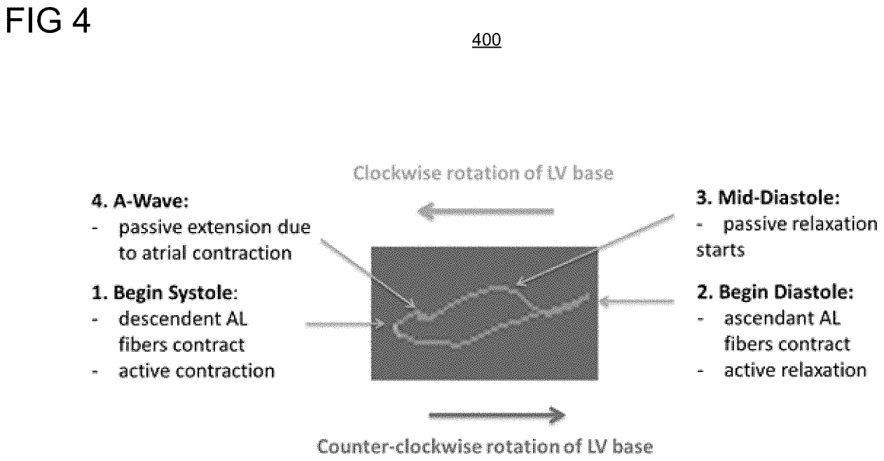

FIG. 4 shows an exemplary endocardial trajectory curve 400. Endocardial trajectory curve 400 may be endocardial trajectory curve 204 shown in FIG. 2. Trajectory curve 400 has distinct geometric regions each characterizing a cardiac event. As shown in FIG. 4, endocardial trajectory curve 400 includes the following geometric regions: Region 1 representing a beginning of ventricular systole where descendent apical loop (AL) fibers contract and the left ventricle is in active contraction, Region 2 representing a beginning of ventricular diastole where ascendant AL fibers contract and the left ventricle is in active relaxation, Region 3 representing mid-diastole where passive relaxation starts in the left ventricle, and Region 4 representing A-wave where the left ventricle is in passive extension due to atrial contraction. It should be understood that regions of endocardial trajectory curve 400 may be associated with other cardiac events.

Another trajectory component of interest is a measure of the angular rotation of a cardiac wall point (endocardial, myocardial, or epicardial) over a cardiac cycle. The angular rotation can be measured from any given point (e.g., the center of the blood pool for the given slice, or the barycenter of the endocardial points for the given slice, or simply the diastolic beginning point) to the current trajectory point. In FIG. 4, for example, the angular rotation as measured from the diastolic beginning point is 0 at the beginning of systole and diastole, mostly negative during systole (shown as a “counterclockwise” rotation in FIG. 4), and mostly positive during diastole (shown as a “clockwise” rotation in FIG. 4).

The regions of the one or more trajectory curves may be identified using any suitable approach. In one embodiment, the regions are automatically identified using, e.g., a suitable algorithm or a machine learning model. For example an “end-of-excursion” (EE) point (usually corresponding to end-systole) can be identified using a trajectory point that is closest to the blood pool center, or a trajectory point that is farthest from the starting point, along the average direction of deformation. The starting and the EE point can be used for initial systolic-diastolic tagging. A curvature analysis of the diastolic component of the curve, identifying the number of large-scale changes in curvature, can be used to further separate the rapid inflow, diastasis, and atrial systolic regions. For robustness, the curvature analysis can be performed on a low-pass (smoothed) version of the trajectory curve. In other embodiments, the regions may be manually identified by, and received as user input from, a user such as, e.g., a clinician. A machine learning algorithm, for example a convolutional neural network (CNN), can be trained on such manual labelling/identification to produce automatic labelling.

At step 306, features of interest associated with the identified regions of the one or more trajectory curves are determined. An exemplary table 208 in FIG. 2 shows values of features of interest associated with each of the regions 206. The features of interest shown in table 208 include total excursion, relative excursion, curvature, and moment. However, it should be understood that the features of interest may include any suitable feature of interest. In one embodiment, the features of interest include spatio-temporal features. For example, the features of interest may include trajectory-specific geometric measures, such as, e.g., pointwise, region-wise, or global excursion (total or relative), curvature, moment, or variability of such measures with respect to spatial position, time stamp, or other variables. In another example, the features of interest may include anatomical measures, such as, e.g., measures of ventricular twist and torsion (e.g., axial, between endocardium and epicardium, longitudinal, between base and apex, etc.). In another example, the features of interest may be based on imaging information, such as, e.g., T1, T2, or perfusion values from MRI images.

The features of interest may be determined using any suitable approach. In one embodiment, the features of interest may be automatically determined using, e.g., a suitable algorithm or a machine learning model. As an example, the total length of the curve can be estimated by summing up the lengths of the individual segments, thus obtaining the total excursion. Cardiac stage excursions can be computed from the labelled trajectory stages. In other embodiments, the features of interest may be manually determined by, and received as user input from, a user such as, e.g., a clinician.

At step 308, a correspondence map is generated by mapping the one or more trajectory curves and/or the determined features of interest to clinical parameters. The correspondence map may be the exemplary correspondence map 210 shown in FIG. 2. The geometric shapes in correspondence map 210 represent data (e.g., data in table 208) illustrating that the map progresses from features based on any number of patients to specific clinical parameters. It should be understood that the correspondence map may be of any suitable form (e.g., a table, a rule-based map, a parametric model, a machine learned map, etc.).

The clinical parameters may include, e.g., functional patient parameters, medical procedure parameters, medical image quality parameters, or any other suitable parameter of interest. Examples of functional patient parameters include ejection fraction (EF), global longitudinal strain (GLS), global circumferential strain (GCS), radial strain, American Heart Association (AHA) regional strain measures, systolic stretch index (SSI), New York Heart Association (NYHA) classes, health state, AHA region ischemic score, medial torsion, and A-wave presence. Examples of medical image quality parameters include signal-to-noise ratio (SNR), signal strength (SS), and quality index (QI). Correspondence map 210 shows the following clinical parameters: EF, GLS, SSI, NYHA classes, health state, AHA region ischemic score, scan quality, medial torsion, and A-wave presence. In one embodiment, the parameters that are included in correspondence map 210 are identified or defined by, and received as user input from, a user such as, e.g., a clinician.

The one or more trajectory curves and/or the determined features of interest are mapped with clinical parameters based on, e.g., a statistical mapping, or machine learning mapping, or any other suitable approach. In one embodiment, the clinical parameters are directly correlated with values of the features of interest. For example, total excursion has a linear relationship with NYHA class such that a value of the total excursion may be associated with an NYHA class. In another example, features of interest are mapped to a medical image quality parameter (e.g., a scan quality index) based on values of the features of interest being within a predefined range. The medical image quality parameter may comprise standard image quality parameters, such as, e.g., SNR, SS, or composite image quality parameters, such as, e.g., the QI. The QI is the product of the intensity ratio (IR) and the tissue signal ratio (TSR). The features of interest may be mapped to the medical image quality parameter using a trained machine learning model for fast computation of the mapping.

In another embodiment, features of interest are mapped to the clinical parameters by computing various measures from the features of interest. For example, a torsion or twist (e.g., axial, between endocardium and epicardium, longitudinal, or between base and apex) may be determined based on a relative location of relevant portions of the heart (e.g., using an image analysis tool that estimates such locations) to help quantitatively evaluate the functional health of the heart. This may be done regionally, thus enabling a more accurate intervention planning and clinical analysis.

In another embodiment, the features of interest are mapped to the clinical parameters by computing surrogate measures related to other clinically relevant regional measures (e.g., AHA-segment strains, systolic pre-stretch in the lateral wall, systolic rebound stretch in the septum, apical excursion or rate of excursion, regional integration of trajectory curves properties like excursion, curvature, moment, etc.) from the features of interest.

At step 310, the correspondence map is output. For example, the correspondence map can be output by displaying the correspondence map on a display device of a computer system, storing the correspondence map on a memory or storage of a computer system, or by transmitting the correspondence map to a remote computer system.

In one embodiment, a clinical decision (e.g., by a clinician) may be made based on the correspondence map. The clinical decision may relate to, e.g., medical image quality evaluation, patient stratification, and diagnostic, planning, or intervention support, etc. For example, as shown as clinical decision 212 in FIG. 2, a clinician may determine whether to re-acquire a medical image with updated protocols based on a medical image quality parameter of the correspondence map.

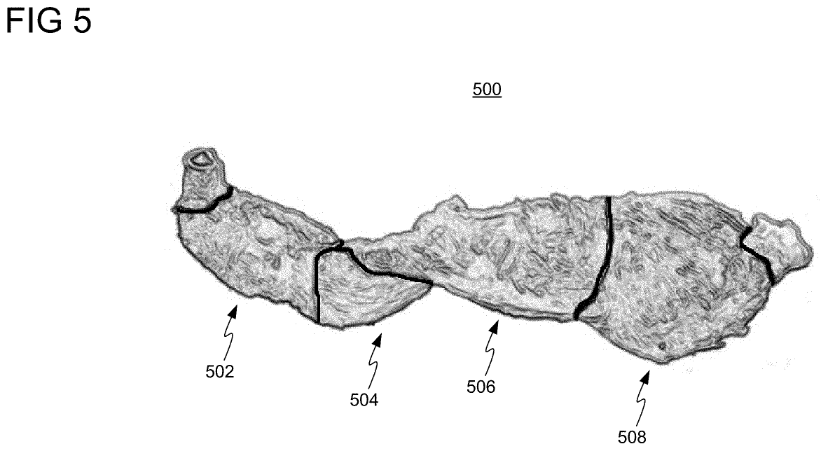

In one embodiment, the clinical parameters may be visualized on a heart unravelling image (e.g., as visible in a stretched-out ventricular myocardial band). FIG. 5 shows a heart unravelling image 500. Heart unravelling image 500 is a visual representation of an anatomically consistent unravelling of a heart into two dimensional portions. Heart unravelling image 500 allows a specific understanding of the coherence and mutual coupling of form and function of the ventricular myocardium. Specifically, the architectural organization plan of the ventricular myocardial fibers, as represented by heart unravelling image 500, describes two spirals in space delimiting a helicoid, where the two cardiac ventricular cavities are nestled. The first spiral is designated as the basal loop (BL) and the second spiral is designated as the apical loop (AL). In both loops, it is possible to distinguish between two consecutive segments. The basal loop includes right segment 502, corresponding to the right ventricle free wall, and left segment 504, corresponding at least in part to the left ventricle free wall. The apical loop includes descendent segment 506 with fibers coming down from the ventricular base to the apex and ascendant segment 508 with fibers from the apex to the base of the heart.

In view of heart unravelling image 500, clinical parameters can be determined that characterize cardiac contraction efficiency, strength, coherence, etc. For example, a coherence score may be computed that penalizes out-of-sync contractions, in which neighboring regions contract with high delay or where remote regions (e.g., right segment 502 and ascendant segment 508) contract with small delay. In accordance with embodiments of the invention, heart unravelling image 500 may visually represent clinical parameters.

FIG. 6 shows a heart unravelling image 600, in accordance with one or more embodiments. As shown in FIG. 6, trajectory curve features or properties are mapped to respective locations on heart unravelling image 600. The trajectory curves represent cardiac movement at the respective location for a full cardiac cycle. It should be understood that other parameters may also be mapped to heart unravelling image 600. The parameters may be related to spatial or temporal properties of the trajectory curves, or to the underlying imaging data.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 2-3. Certain steps or functions of the methods and workflows described herein, including one or more of the steps or functions of FIGS. 2-3, may be performed by a server or by another processor in a network-based cloud-computing system. Certain steps or functions of the methods and workflows described herein, including one or more of the steps of FIGS. 2-3, may be performed by a client computer in a network-based cloud computing system. The steps or functions of the methods and workflows described herein, including one or more of the steps of FIGS. 2-3, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 2-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 702 that may be used to implement systems, apparatus, and methods described herein is depicted in FIG. 7. Computer 702 includes a processor 704 operatively coupled to a data storage device 712 and a memory 710. Processor 704 controls the overall operation of computer 702 by executing computer program instructions that define such operations. The computer program instructions may be stored in data storage device 712, or other computer readable medium, and loaded into memory 710 when execution of the computer program instructions is desired. Thus, the method and workflow steps or functions of FIGS. 2-3 can be defined by the computer program instructions stored in memory 710 and/or data storage device 712 and controlled by processor 704 executing the computer program instructions. For example, the computer program instructions can be implemented as computer executable code programmed by one skilled in the art to perform the method and workflow steps or functions of FIGS. 2-3. Accordingly, by executing the computer program instructions, the processor 704 executes the method and workflow steps or functions of FIGS. 2-3. Computer 704 may also include one or more network interfaces 706 for communicating with other devices via a network. Computer 702 may also include one or more input/output devices 708 that enable user interaction with computer 702 (e.g., display, keyboard, mouse, speakers, buttons, etc.).

Processor 704 may include both general and special purpose microprocessors, and may be the sole processor or one of multiple processors of computer 702. Processor 704 may include one or more central processing units (CPUs), for example. Processor 704, data storage device 712, and/or memory 710 may include, be supplemented by, or incorporated in, one or more application-specific integrated circuits (ASICs) and/or one or more field programmable gate arrays (FPGAs).

Data storage device 712 and memory 710 each include a tangible non-transitory computer readable storage medium. Data storage device 712, and memory 710, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 708 may include peripherals, such as a printer, scanner, display screen, etc. For example, input/output devices 708 may include a display device such as a cathode ray tube (CRT) or liquid crystal display (LCD) monitor for displaying information to the user, a keyboard, and a pointing device such as a mouse or a trackball by which the user can provide input to computer 702.

An image acquisition device 714 can be connected to the computer 702 to input image data (e.g., medical images) to the computer 702. It is possible to implement the image acquisition device 714 and the computer 702 as one device. It is also possible that the image acquisition device 714 and the computer 702 communicate wirelessly through a network. In a possible embodiment, the computer 702 can be located remotely with respect to the image acquisition device 714.

Any or all of the systems and apparatus discussed herein may be implemented using one or more computers such as computer 702.

One skilled in the art will recognize that an implementation of an actual computer or computer system may have other structures and may contain other components as well, and that FIG. 7 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method comprising:
identifying regions of one or more trajectory curves that correspond to cardiac events, the one or more trajectory curves representing cardiac movement;
determining features of interest associated with the identified regions; and
generating a correspondence map by mapping the determined features of interest to clinical parameters.

2. The method of claim 1, wherein the one or more trajectory curves comprise an endocardial trajectory curve, a myocardial trajectory curve, and an epicardial trajectory curve.

3. The method of claim 1, wherein identifying regions of one or more trajectory curves that correspond to cardiac events comprises:
identifying geometric regions of the one or more trajectory curves that correspond to a beginning of systole, a beginning a diastole, a middle of diastole, and an A-wave.

4. The method of claim 1, wherein the features of interest comprise geometric measures of the identified regions of the one or more trajectory curves and anatomical measures of the identified regions of the one or more trajectory curves.

5. The method of claim 1, wherein generating a correspondence map by mapping the determined features of interest to clinical parameters comprises:
mapping the determined features of interest to the clinical parameters based on a statistical mapping of the determined features of interest to the clinical parameters.

6. The method of claim 5, wherein mapping the determined features of interest to the clinical parameters based on a statistical mapping of the determined features of interest to the clinical parameters comprises:
mapping the determined features of interest to the clinical parameters based on a correlation between the determined features of interest and the clinical parameters.

7. The method of claim 1, wherein generating a correspondence map by mapping the determined features of interest to clinical parameters comprises:
mapping the determined features of interest to the clinical parameters using a machine learning model.

8. The method of claim 1, wherein the correspondence map is used for clinical decision making.

9. The method of claim 1, further comprising:
visually representing the clinical parameters on a heart unravelling image.

10. An apparatus comprising:
means for identifying regions of one or more trajectory curves that correspond to cardiac events, the one or more trajectory curves representing cardiac movement;
means for determining features of interest associated with the identified regions; and
means for generating a correspondence map by mapping the determined features of interest to clinical parameters.

11. The apparatus of claim 10, wherein the one or more trajectory curves comprise an endocardial trajectory curve, a myocardial trajectory curve, and an epicardial trajectory curve.

12. The apparatus of claim 10, wherein the means for identifying regions of one or more trajectory curves that correspond to cardiac events comprises:
means for identifying geometric regions of the one or more trajectory curves that correspond to a beginning of systole, a beginning a diastole, a middle of diastole, and an A-wave.

13. The apparatus of claim 10, wherein the features of interest comprise geometric measures of the identified regions of the one or more trajectory curves and anatomical measures of the identified regions of the one or more trajectory curves.

14. The apparatus of claim 10, further comprising:
means for visually representing the clinical parameters on a heart unravelling image.

15. A non-transitory computer readable medium storing computer program instructions, the computer program instructions when executed by a processor cause the processor to perform operations comprising:
identifying regions of one or more trajectory curves that correspond to cardiac events, the one or more trajectory curves representing cardiac movement;
determining features of interest associated with the identified regions; and
generating a correspondence map by mapping the determined features of interest to clinical parameters.

16. The non-transitory computer readable medium of claim 15, wherein generating a correspondence map by mapping the determined features of interest to clinical parameters comprises:
mapping the determined features of interest to the clinical parameters based on a statistical mapping of the determined features of interest to the clinical parameters.

17. The non-transitory computer readable medium of claim 16, wherein mapping the determined features of interest to the clinical parameters based on a statistical mapping of the determined features of interest to the clinical parameters comprises:
mapping the determined features of interest to the clinical parameters based on a correlation between the determined features of interest and the clinical parameters.

18. The non-transitory computer readable medium of claim 15, wherein generating a correspondence map by mapping the determined features of interest to clinical parameters comprises:
mapping the determined features of interest to the clinical parameters using a machine learning model.

19. The non-transitory computer readable medium of claim 15, wherein the correspondence map is used for clinical decision making.

20. The non-transitory computer readable medium of claim 15, the operations further comprising:
visually representing the clinical parameters on a heart unravelling image.
